(19) United States
(12) Patent Application Publication
(10) Pub. No.: US 2008/0227748 A1
(43) Pub. Date: Sep. 18, 2008

(54) Medicament Made From Laminarine or Oligolaminarines For Treatment of Septicemia and Septic Shock

(75) Inventors: Jean-Claude Yvin, Saint Malo (FR); Nathalie Delzenne, Genval (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: LABORATOIRES GOEMAR, Saint Malo (FR)

(21) Appl. No.: 11/914,703

(22) PCT Filed: May 18, 2006
(86) PCT No.: PCT/FR06/01129

§ 371 Date: May 22, 2008

(30) Foreign Application Priority Data
May 18, 2005 (FR) 0504983

(51) Int. Cl. A61K 31/715; A61P 7/00

(52) U.S. Cl. 514/54

(57) ABSTRACT

The invention relates Lo the use of laminarine, preferably in the soluble form thereof or an oligolaminarine, for the production of a medicament for treatment of septicemia and septic shock.

The invention relates to a medicament based on laminarin or on oligolaminarins which is suitable for the treatment of septicemia and septic shock.

Septicemia or sepsis is characterized by an excessive reaction of the immune system and coagulation system of the body due to an infection. This reaction is marked by a generalized infection, that is to say of the entire body, and by clotting of the blood, phenomenon which can rapidly result in failure of an organ, then called severe sepsis, and frequently death.

Severe sepsis is thus defined as sepsis accompanied by the failure of one or more organs, such as, for example, the lungs, kidneys, cardiovascular system and/or other organs.

Septic shock is a common and serious form of severe sepsis. It occurs when the cardiovascular system begins to fail and when the arterial pressure falls to a dangerously low level.

The symptoms of sepsis can vary from one person to another according to age, previous health and the reaction of the patient to the infection. The progress of the disease cannot be predicted, the condition of some patients rapidly deteriorating and passing to the stage of severe sepsis, these patients then suffering from failure of the lungs, kidneys, cardiovascular system and/or other organs.

Sepsis is generally the result of a bacterial, viral or fungal infection which can occur in any part of the body.

People suffering from pneumonia, trauma, a surgical operation, burns or a serious disease, such as cancer, may be predisposed to sepsis; people who die following complications of cancer or of pneumonia often in reality die from sepsis. Elderly people and patients whose immune system is weakened by medicaments are particularly vulnerable.

Mortality can reach 30 to 70% of cases.

The number of cases of sepsis or a septic shock caused by gram+ or gram− bacteria, fungi, viruses and parasites is showing a worrying increase probably related to
the increase in the use in the hospital environment of catheters and other invasive devices,
the increase in chemotherapy, and also the increased use of immunosuppressants in the case of organ transplants,
the increase in serious inflammatory diseases.

Sepsis and septic shock are treated by the administration
of fluids composed of glucose-enriched physiological sera,
of vasoconstrictive agents, in order to restore the blood pressure, and
of antibiotics.

These treatments also comprise oxygenation of the patients.

The conventional treatments just referred to are currently supplemented by alternative approaches targeted at suppressing the deleterious effects of the inflammation and at stimulating antimicrobial defenses, including in particular the administration
of anti-LPS antibodies,
of anti-TNF-α antibodies
of novel anticoagulants, namely active protein C and drotecogin.

The aim of the invention is, in particular, to provide the medical world with an additional means of combating septicemia and septic shock.

In point of fact, it has been found that the clinical phenomena which precede the development of septicemia or septic shock bring about a very broad activation of the cell responses, the consequence of which is a release, into the serum and into the liver, of a whole range of inflammatory mediators, such as cytokines and in particular the cytokine or chemokine TNF-α (Tumor Necrosis Factor-α), prostaglandins, lipid mediators and activated oxygen entities, which will keep a number of organs inflamed, the most affected among which are the kidneys, liver, intestines and lungs.

The reasoning on which the invention is based was thus to find a means which makes it possible to reduce the content of inflammatory mediators quoted above, in particular TNF-α, in the serum of patients and to increase the number of Kupffer cells, the reaction of which is known in particular in reinforcing immunity and combating pathogens.

It is to the credit of the Appellant Company to have found that, surprisingly and unexpectedly, the administration to the patient, in particular by the oral route, of an effective amount of known β-1,3-glucans, being a matter of laminarin, in particular in its soluble form, and of oligolaminarins, makes it possible to act, in the direction of a reduction, on the concentration of TNF-α and to act, in the direction of an increase, on the number of Kupffer cells.

An object of the invention is thus the use of laminarin, preferably in its soluble form, or of oligolaminarins in the preparation of a medicament intended for the treatment of septicemia and septic shock.

According to an advantageous embodiment, an object of the invention is the use of laminarin or of oligolaminarins in the preparation of a medicament, for oral administration, intended for the treatment of septicemia or sepsis or septic shock, said medicament being administered to the patient in an amount sufficient to contribute daily to the body an amount of laminarin or of oligolaminarins of 2 to 21 mg, preferably of 6 to 15 mg, per kg of bodyweight.

Before describing the studies which have allowed the Appellant Company to arrive at the invention, it should be remembered that the term “laminarin” denotes mixtures of polysaccharides of β-1,3-glucan type which are extracted from brown algae and which have a molecular weight from approximately 2500 to approximately 6000.

For their part, oligolaminarins are a mixture of oligosaccharides of β-glucan type resulting from the hydrolysis of laminarin which is characterized by a degree of polymerization of between 2 and 10, that is to say a molecular weight of between 350 and 2000.

Laminarin is composed of a linear main chain of 15 to 35 glucopyranose units joined via β-1,3 acetal bonds, this main chain carrying a low proportion of branchings.

The average degree of polymerization is approximately 25.

Laminarin exists in a soluble form and in an insoluble form.

To extract it from brown algae and to separate, as required, the soluble and insoluble forms of laminarin, reference may be made in particular to the international patent application WO 03/045 414 of the Applicant Company, published on 5 Jun. 2003.

The specific laminarin which was used in the context of the studies described below and which made it possible to arrive at the invention has a molecular weight of 4500 and an average degree of polymerization of 25 and is composed essentially of polysaccharides with degrees of polymerization ranging from 20 to 30.

It was provided in the form of a odorless and flavorless off-white powder.

The experimental studies discussed above made it possible to show that laminarin, administered in particular orally, exerts a protective effect with regard to the repercussions on the body of an endotoxic shock.

Due to these results, it may reasonably be believed that laminarin exerts an immunostimulant effect on immunocompetent cells, such as macrophages and NK (natural killer) cells.

In the context of the studies in question, the endotoxic shock created in the organisms subjected to the experiments was brought about by the administration of lipopolysaccharides or LPSs, the effect of which is similar, as is known, to a bacterial attack.

The animals selected for the abovesaid studies were male Wistar Han rats (Harlem, Netherlands).

The experiments were carried out on two groups of eight rats each.

The rats of one of the two groups—“control group”—had available to them “ad libitum” a standard feed with the AO4 trade name (UAR, France); the rats of the other group had available to them the same feed but supplemented with the above said laminarin, sold under the “Dectinan” trade name; in a first phase and for four days (−4 to −1), the feed was enriched with 5% by weight of Dectinan and then with 10% by weight in a second phase of 21 days (days 0 to 21); this second group of rats is denoted by “Dectinan group”.

On the 21st day, endotoxic shock was brought about with regard to the rats in the two groups by intraperitoneal injection of LPS in an amount corresponding to 10 mg per kg of bodyweight.

On days 22 and 23, the rats were deprived of feed and water; they were sacrificed on day 24.

The following parameters were studied:
the consumption of water and of feed
the change in the bodyweight
the lipid and glucide balance
the rectal temperature
the number of leukocytes
the inflammatory mediators (serum TNF-α and PGE₂ or prostaglandin E₂)
the histological markers of the resident macrophages of the liver, that is to say the Kupffer cells (ED-2 and/or peroxidase activity)
the markers of toxicity of the LPS treatment (serum ALT, AST and LDH activities; histology of the liver, spleen, thymus and lungs)
the cecocolic fermentation (weight of the feces and cecal proliferation)

The days on which the various withdrawals and measurements are made are identified in table I below.

TABLE I

| Dectinan | 5% Dectinan | | | | 10% Dectinan | | | | | | | | | | | | | | | | | | | | | | Deprived | |
|---|---|---|---|---|---|---|---|---|---|---|---|---|---|---|---|---|---|---|---|---|---|---|---|---|---|---|---|---|
| Group | | | | | | | | | | | | | | | | | | | | | | | | | | | of feed | |
| | | | | | | | | | | | | | | | | | | | | | | | | | | | and | |
| | | | | | | | | | | | | | | | | | | | | | | | | | | | water | |
| Control | Standard diet | | | | | | | | | | | | | | | | | | | | | | | | | | Deprived | |
| Group | | | | | | | | | | | | | | | | | | | | | | | | | | | of feed | |
| | | | | | | | | | | | | | | | | | | | | | | | | | | | and | |
| | | | | | | | | | | | | | | | | | | | | | | | | | | | water | |
| Days | −4 | −3 | −2 | −1 | 0 | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
| Taking of | X | | | | X | | | | | | | | X | | | | | | | X | | | | | | | X | |
| blood | | | | | | | | | | | | | | | | | | | | | | | | | | | | |
| Collecting | | | | | X | | | | | | | | X | | | | | | | X | | | | | | | X | |
| of feces | | | | | | | | | | | | | | | | | | | | | | | | | | | | |
| Taking of | X | X | | | X | X | X | X | | | | X | X | | X | X | | | X | X | | X | X | | | X | X | X |
| bodyweight | | | | | | | | | | | | | | | | | | | | | | | | | | | | |
| Water/diet | X | X | | | X | X | X | X | | | | X | X | | X | X | | | X | X | | X | X | | | X | X | |
| consumption | | | | | | | | | | | | | | | | | | | | | | | | | | | | |

More particularly, the following determinations were carried out:

a) Before injection of LPS
bodyweight
weight of wet feces and feces after freeze drying (collected over 24 h)
weight of the diet and of the drinking water
taking of blood from the caudal vein: quantitative determination of triglycerides and glucose

b) After injection of LPS (at hours 0, 2, 4, 6, 8 and 24)
rectal temperature
taking of blood in the caudal vein: quantitative determination of triglycerides, glucose, PGE₂, TNF-α, free fatty acids, ALT, AST and LDH (lactase dehydrogenase) activities

c) At the time of sacrifice:
blood from the portal vein: quantitative determination of free fatty acid
blood from the vena cava: determination of the blood formula (cell counting, hematocrit and hemoglobin) and quantitative determination of free fatty acids, triglycerides, PGE₂, TNF-α and ALT, AST and LDH activities in the serum
liver: weight, freezing of samples for histological studies (hematoxylin-eosin, detection of Kupffer cells via the peroxidase activity and the ED-2 antigen)
spleen: weight and preservation of samples in formaldehyde for histological studies (hematoxylin-eosin)
cecum: weight of full cecum and of empty cecum
thymus and lungs: samples preserved in formaldehyde for a histological study (hematoxylin-eosin)

The results recorded were subjected to a statistical treatment.

In this regard, the values mentioned later represent the mean ±SDM (Standard Deviation of the Mean). The results obtained before the administration of LPS and at the time of sacrifice were analyzed with the Student's t-test (described in: Appareils et Methodes en Biochimie [Equipment and Methods in Biochemistry] by Pierre Kamoun, 3rd edition, medicine sciences Flammarion, 1987) between the two groups using the Excell program (described in More Excell html +intro java +cd, author(s) Gottleber, 01-2000). The kinetic studies of the various parameters measured after the injection of LPS were processed by a 2-way Anova analysis: analysis of variance, described in “For the Behavioral Sciences Researcher” by Rudolph N. Cardinal, published by Lawrence, via the SPSS program (described in Traitement de données avec SPSS pour Windows [Data Processing with SPSS for Windows] (edition 8.0, 9.0), author(s): Fernando Ouellet and Gerald Baillargeon, publication date: 01-2000).

A logarithmic transformation of the individual values was carried out for the TNF-α and PGE₂ parameters before the statistical treatment. The differences are regarded as statistically significant when p<0.05 (p meaning probability with a tolerance of 5%).

The results that are obtained before the administration of LPS are as indicated below:

One week after their arrival, the rats had a mean weight of 136±1 g and were distributed in randomized fashion into 2 groups of 8 animals: the control group and the Dectinan® group. The gain in bodyweight and the total consumptions of water and of feed from the first day of treatment up to the day of injection of LPS are shown in table 1. The diet had no effect on these 3 parameters, which is evidence of the absence of toxicity of laminarin.

The data recorded have been combined in table II below:

TABLE II
Gain in weight and consumption

| | Control | Dectinan ® |
|---|---|---|
| Gain in weight (g) | 125 ± 3 | 125 ± 3 |
| Total consumption of feed (g) | 449.5 ± 12.3 | 431.5 ± 6.0 |
| Total consumption of drinking | 576.9 ± 13.0 | 611.5 ± 17.9 |
| water (ml) | | |

n = 8.
p > 0.05 Students's t-test

n=8. p>0.05 Student's t-test

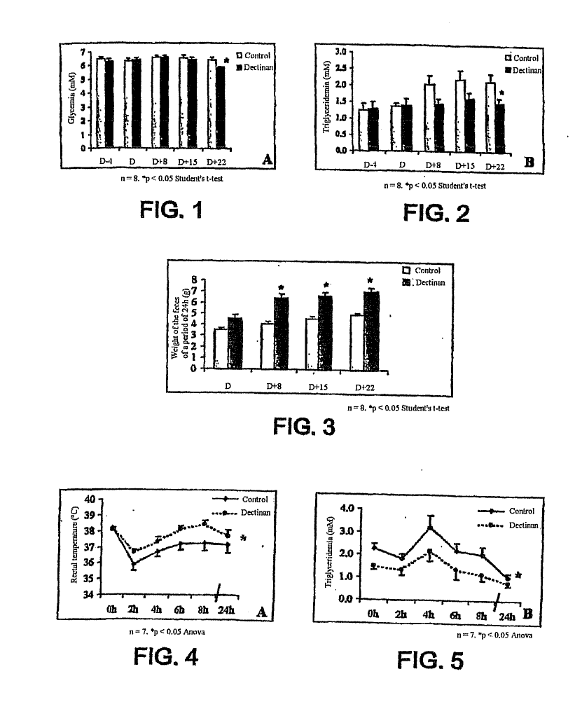

Blood samples were taken weekly in order to monitor the change in glycemia and triglyceridemia during the feeding treatment. The diet enriched in Dectinan® significantly reduces glycemia and triglyceridemia after 25 days of treatment (4 days at 5% and 21 days at 10%), as results respectively from FIGS. 1 and 2, which illustrate the change of glycemia and in triglyceridemia during the treatment.

The feces of a period of 24 hours were collected and weighed once weekly.

As results from the table shown in FIG. 3, the diet enriched in Dectinan® increases the fecal weight from the first week of treatment at 10%.

The loss of water in the feces is also greater; in the third week of administration of feed enriched in Dectinan® at 10%, the percentage of water of the feces is 24.7±1.4%, whereas it is 17.8±1.5% for the control group (*p<0.05, Student's t-test).

The LPS induces a transitory hyperthermia and a transitory hyperglycemia 2 hours after its administration. The diet enriched in Dectinan® significantly enhances these two phenomena. In addition, the rats of the Dectinan group maintain a higher temperature than the rats of the control group after the time of sacrifice.

The prior treatment for 25 days with Dectinan® mixed with the feed prevents, or at least lessens, the metabolic effects resulting from acute inflammation generated by the LPS. In other words, Dectinan® reduces the hypothermia, the hypertriglyceridemia and the hyperglycemia of the first hours which form the injection of LPS, as result from the curves appearing in the graphs of FIGS. 4 (hyperthermia), 5 (hypertriglyceridemia) and 6 (hyperglycemia).

In other words, the administration of feed enriched in Dectinan® protects the liver against the toxic effect related to acute inflammation mimicked by the injection of LPS, a phenomenon which goes hand in hand with a modulation of the immune cells of the liver.

Furthermore, the administration of this feed counteracts the increase in the specific marker of liver distress, this marker being composed of the serum activity of alanine aminotransferase (ALT); it also corrects other serum parameters which may reflect detrimental tissue changes in some of the organs, including the liver, namely the aspartate amino-transferase (AST) and lactate dehydrogenase (LDH) activities.

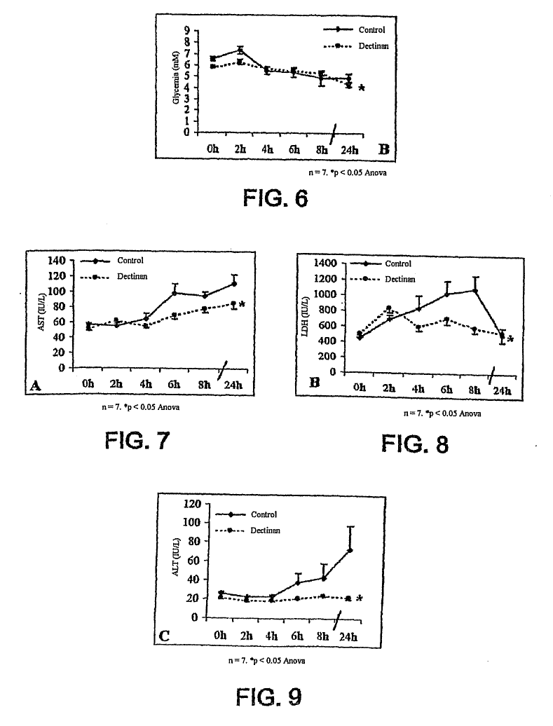

The results recorded are reflected by the curves shown in FIGS. 7 (change in AST), 8 (change in LDH) and 9 (change in ALT).

The following concentrations in the serum were also measured (in pg/ml):
of the cytokine TNF-α at times 2 hours, 4 hours, 6 hours, 8 hours and 24 hours after the administration of LPS;
a prostaglandin PGE₂ at times 2 hours and 24 hours after the administration of LPS.

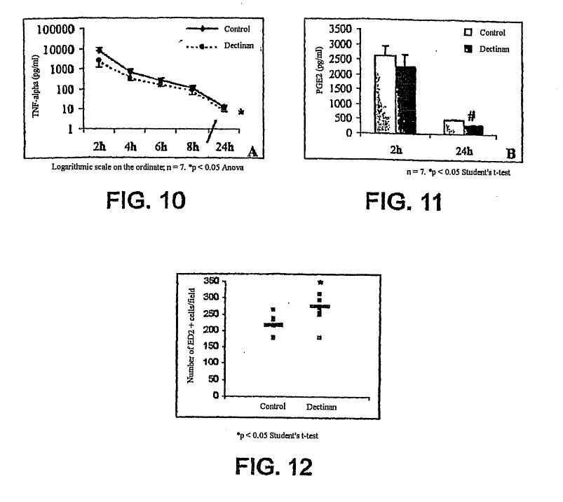

The results are given respectively in the graphs of FIGS. 10 (TNF-α) and 11 (PGE₂).

The TNF-α is not detectable at times 0 hour due to the absence of inflammatory stimulus. Furthermore, it is noted that the presence of Dectinan in the feed reduces the level of TNF-α and of PGE₂ in the serum of rats subjected to endotoxic shock.

The rats were deprived of feed and water after the administration of LPS in order to avoid problems of interpretation related to the anorexigenic effect of this treatment.

After sacrificing, the various weighings, the results of which are combined in table III, were carried out.

TABLE III
Bodyweight and weight of the organs

| | Control | Dectinan ® |
|---|---|---|
| Bodyweight (g) | 239 ± 4 | 234 ± 3 |
| Weight of the body organs (%) | | |
| liver | 3.56 ± 0.13 | 3.88 ± 0.08≠ |
| spleen | 0.36 ± 0.02 | 0.35 ± 0.02 |
| empty cecum | 0.31 ± 0.02 | 0.47 ± 0.01* |

n = 7.
≠p = 0.07;
*p < 0.05 Student's t-test

The following observations may be drawn from the examination of the results combined in table III. The loss in weight is not significantly modified by the administration of the feed comprising Dectinan® (25±2 g versus 22±1 g; with respect to the bodyweight). The weight of the animals is not different between the 2 groups after depriving of feed and water for 24 hours. The weight of the liver increases slightly in the Dectinan® group with a p value close to significant. The diet enriched in Dectinan® brings about significant cecal proliferation, as is attested by the mean values of the weight of the cecal tissue and of the cecal contents (3.2±0.32 g for the Dectinan® group versus 2.15±0.24 g for controls *p<0.05 Student's t-test).

The erythrocytes and leucocytes were also counted and different blood parameters were also measured, which operations were carried out on the blood samples from the vena cava.

The results are combined in table IV.

TABLE IV
Blood formula

| | Control | Dectinan ® |
|---|---|---|
| Leucocytes (×1000/mm³) | 3.59 ± 0.14 | 4.02 ± 0.28 |
| monocytes (%) | 2.74 ± 0.17 | 2.14 ± 0.19* |
| lymphocytes (%) | 33.12 ± 2.23 | 31.67 ± 2.42 |
| granulocytes (%) | 64.15 ± 2.13 | 66.18 ± 2.39 |
| Erythrocytes (×1000/mm³) | 7.38 ± 0.13 | 7.31 ± 0.20 |
| Hematocrit (%) | 43.7 ± 1.0 | 42.6 ± 1.0 |
| Hemoglobin (g/dl) | 13.6 ± 0.3 | 13.3 ± 0.3 |

n = 7.
*p < 0.05 Student's t-test

On examining the results combined in table IV, it is found that only the percentage of monocytes among the leucocytes is significantly different; it is lower in the rats fed with a diet enriched in Dectinan® in comparison with rats of the control group.

The detection of the membrane antigen ED2 by an immuno-histochemical technique makes it possible for the specific detection of the Kupffer cells in the liver. These cells are situated in particular in the portal spaces; they are rounder and more swollen 24 hours after the administration of LPS. In order to refine the observation of the liver sections, a quantitative approach was carried out: 3 fields of 10×10 squares (comprising at least 3 vessels belonging to a portal space) were selected per liver section in order to count the ED2-positive cells. Pretreatment with a diet enriched in Dectinan® increases the number of liver macrophages (259±19 ED2+ cells/field versus 217+14 ED2+ cells/field, n=6; p=0.1, Student's t-test; these resident macrophages of the liver purify the liver from the circulating lipopolysaccharides. When the abnormally low value of rat 8 (represented by a blank square), belonging to the Dectinan® group, is not included in the mean, the difference is significant.

The result of the abovesaid measurements is illustrated in FIG. 12, which shows the quantification of the Kupffer cells by means of the detection of ED2 in the liver sections.

It is known that the enzyme peroxidase is present within macrophages (such as Kupffer cells), neutrophils and eosinophils. The administration of LPS increases the number of positive cells in the liver as immune cells (neutrophils) are recruited. The diet supplemented with Dectinan® appears to reduce the number of positive cells.

The histological analysis after staining with eosin and with hematoxylin reveals the architecture of the tissues and also the distressed cells or the foci of necrosis. In the liver, the detrimental changes, mainly necrotic foci, related to the injection of LPS are virtually invisible, both in the Dectinan® group and in the control group; it is noted that the detrimental biochemical changes (mainly the increase in the ALT) are generally earlier and are not necessarily reflected by detrimental changes in the liver tissue. The lung exhibits edemas typically related to the inflammatory state, whereas the thymus and the spleen reveal necrotic regions.

The combined results above reflect the protective effects of Dectinan® with respect to the increase in the AST, LDH and ALT activities in the serum. It is observed that the toxicity of the LPS is lower due to the protective effect; it is correlated with a reduced secretion of TNF-α, which is a proinflammatory cytokine. A decrease in the concentration of the PGE₂ 24 hours after the inflammatory stimulus is also observed.

Hypothermia constitutes an early manifestation after administration of a high dose of LPS. In fact, TNF-α is regarded as a “cryogenic” signal involved in the maintenance of hyperthermia; this phenomenon is more pronounced in the rats of the control group, which have, in this case, higher levels of TNF-α than those of the Dectinan® group.

The endotoxic shock brought about by the administration of LPS also produces a transitory hyperglycemia, due in part to an intense hepatic glycogenolysis, followed by a gradual reduction in glycemia, resulting from the use of the glucose by the peripheral tissues and in particular by the immune cells. As transitory hyperglycemia is less significant in the rats of the Dectinan® group, hepatic glycogenolysis might be evoked as potential metabolic target.

The administration of high doses of LPS induces a hypertriglyceridemia which is encountered in both groups of animals. The levels of serum triglycerides are systematically lower after a diet enriched with Dectinan® but the variations in triglycerides due to injection of LPS are similar. A fall in the levels of triglycerides in the blood, apart from the inflammatory stimulus, constitute a highly advantageous result.

The histological study of the ED2 antigen demonstrates an increase in the number of Kupffer cells under the diet enriched with Dectinan®; it might involve either recruitment of original cells resulting from the marrow or intrahepatic proliferation of existing Kupffer cells. With regard to the histological study of the endogenous peroxidase, which results from the activity of the Kupffer cells and from the immune cells recruited during an inflammatory state, it appears to be less significant after a diet enriched in Dectinan®. Furthermore, it is interesting to observe that the number of circulated monocytes is lower in the Dectinan® group than in the control group after the administration of LPS.

In conclusion, it is interesting to observe that the administration of feed enriched in Dectinan® makes it possible to reduce the inflammation related to the LPS, with favorable consequences with regard to the enzymes of hepatic origin and with regard to the recruitment of the number of immune cells in the liver, this phenomenon going hand in hand with an increase in the number of liver microphages.

The medicament obtained by the use in accordance with the invention of laminarin or oligolaminarins can be provided in the form of tablets, hard gelatin capsules or sachets or also, for example, in a liquid form, in particular in the form of a solution to be taken orally.

### EXAMPLE 1

### Tablets

Uncoated tablets of 450 mg based on Dectinan®:

Their formulation is as follows:

| | |
|---|---|
| 225 mg of Dectinan ® | i.e. 50% |
| 176 mg of maltodextrins | i.e. 39.1% |
| 40 mg of tricalcium phosphate | i.e. 8.9% |
| 9 mg of magnesium stearate | i.e. 2% |

The constituents are sieved, mixed and then compressed in the form of ingots. The latter are subsequently introduced into a mill which converts them into granules with a size suitable for a phase of tableting the final tablet.

The tablets can be sold in the uncoated, film-coated or sugar-coated form. They can be packaged in bottles or in blister packs of 15 or 20 units.

The dosage is from 2 to 4 tablets daily, taken on 2 occasions during meals; to be taken in courses of treatment lasting 15 days.

### EXAMPLE 2

### Hard Gelatin Capsules

Hard gelatin capsules of plant or marine origin of 363 mg based on Dectinan®:

Their formulation is as follows for 288 mg of powder:

| | |
|---|---|
| 225 mg of Dectinan ® | i.e. 78.1% |
| 60 mg of microcrystalline cellulose | i.e. 20.8% |
| 2 mg of silica | i.e. 0.7% |
| 1 mg of magnesium stearate | i.e. 0.4% |

These hard gelatin capsules can be packaged in bottles or in blister packs of 15 or 20 units.

The dosage is from 2 to 4 hard gelatin capsules daily, taken on 2 occasions during meals: to be taken in courses of treatment lasting 15 days.

### EXAMPLE 3

### Sachets

Dose sachets of 1 g based on Dectinan®:

Their formulation is as follows:

| | |
|---|---|
| 225 mg of Dectinan ® | i.e. 22.5% |
| 275 mg of maltodextrins | i.e. 27.5% |
| 295 g of fructose | i.e. 29.5% |
| 200 mg of lemon juice powder | i.e. 20% |
| 5 mg of silica | i.e. 0.5% |

One liter of an aqueous solution comprising 22.5 g of Dectinan® is mixed with 27.5 g of maltodextrins. The other constituents are then incorporated. The solution thus obtained is subsequently atomized and the powder granulated. The powder is subsequently dried at 60° C. overnight, so as to obtain a dry matter of less than 3%. Packaging is subsequently carried out in aluminum sachets with a size of 60×30 mm comprising 1 g of powder.

The dosage is from 2 to 4 sachets daily, taken on 2 occasions during meals; to be taken in courses of treatment lasting 15 days.

### EXAMPLE 4

### Solution to be Taken Orally

| | |
|---|---|
| Water | i.e. 92.85% |
| Dectinan ® | i.e. 2.25% |
| Fructose | i.e. 4% |
| Lemon flavoring | i.e. 0.2% |
| Citric acid | i.e. 0.5% |
| Potassium sorbate | i.e. 0.1% |
| Sodium benzoate | i.e. 0.1% |

This solution to be taken orally can be packaged in singled doses of 10 ml or in a 100 ml bottle.

The dosage is from 2 to 4 doses of 10 ml or 2 to 4 tablespoonfuls daily, taken on 2 occasions during meals; to be taken in courses of treatment lasting 15 days.

What is claimed is:

1. The use of laminarin, preferably in its soluble form, or of oligolaminarins in the preparation of a medicament intended for the treatment of septicemia and septic shock.

2. The use of laminarin or of oligolaminarins in the preparation of a medicament, for oral administration, intended for the treatment of septicemia and septic shock, said medicament being administered to the patient in an amount sufficient to contribute daily to the body an amount of laminarin or of oligolaminarins of 2 to 21 mg, preferably of 6 to 15 mg, per kg of bodyweight.
